(19) United States
(12) Patent Application Publication
(10) Pub. No.: US 2003/0105392 A1
(43) Pub. Date: Jun. 5, 2003

(54) Apparatus for measuring concentration of a specific ingredient in-situ

(76) Inventor: Wei-Kung Wang, Taipei (TW)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/123,124

(22) Filed: Apr. 16, 2002

Related U.S. Application Data
(63) Continuation-in-part of application No. 09/766,237, filed on Jan. 19, 2001.

(51) Int. Cl. A61B 5/05

(52) U.S. Cl. 600/345

(57) ABSTRACT

Disclosed is an apparatus for measuring the concentration of a specific ingredient in a solution. According to one embodiment of the present invention, said apparatus comprises: a signal collector for collecting a plurality of signals emitted from a target in a selected volume of the solution, and one of the signals being corresponding to the selected volume; detectors for detecting the signals; and beam splitters for splitting said signals and transmitting the signals to the detectors. The present invention provides an apparatus for effectively measuring concentration in-situ without the need of extracting the solution out of its original container.

The present application is a continuation-in-part of pending U.S. patent application Ser. No. 09/766,237, entitled “MOLD-IN METHOD AND APPARATUS,” and filed on Jan. 19, 2001 by the same inventor of the present application.

### FIELD OF INVENTION

The present invention relates to an apparatus for measuring the concentration of a specific ingredient in-situ.

### BACKGROUND AND SUMMARY OF INVENTION

To measure the concentration of an ingredient in a solution is usually to have the solution extracted from its container and put into a test tube or a cuvette, which is another container with a known volume (or more precisely, a known signal path). After the specific signal generated from the specific ingredient is measured, together with the known volume, the concentration can be determined by the ratio of the amount of ingredient to the volume.

However, if such measurement is to be taken an in-situ (i.e., the solution had better not be extracted from the container such as the cases of extracting blood from the blood vessel or moving a sample out of a production line), the information about the volume is required to determine the concentration.

Therefore, for the case of measuring the concentration of one ingredient, at least two signals: one for the volume and the other for the specific ingredient, are needed for the concentration measurement. For the case of two ingredients, three signals are needed for the measurement. When there are (N−1) ingredients, by deduction, N signals including one for volume and (N−1) signals for the (N−1) ingredients are needed. In order to separate and determine each of these N signals, usually a grating is used. Then based on the ratio of the signal for each ingredient to the signal for the volume, the concentrations of N ingredient can be obtained.

For the signal of the volume, it can be obtained by a direct measurement of the volume by, for example, ultrasound or light reflection. Then, the length of the signal path can be determined. According to one aspect of the present invention, the specific signal from the solvent is measured, instead of measuring the signal of the volume. Because the solvent constitutes most of the volume in the solution, based on the signal of the solvent, the volume of the effective container can be determined even if the container does not have a well-defined shape. Besides the solvent, a marker with known concentration could also be used to determine the volume, and the signal of the marker is regarded as the signal of the volume. Such a marker could be either the intrinsic type or the added-in type (explained in detail below).

### BRIEF DESCRIPTION OF DRAWING

The present invention can be better understood through the accompanying drawing in which:

FIG. 1 shows an apparatus for measuring the concentration of a specific ingredient in-situ, according to one embodiment of the present invention.

### DETAILED DESCRIPTION OF INVENTION

In the figured embodiment, an optical signal (enamation or induced signal) is used as an example.

To measure glucose in the blood vessel in a human body, the glucose signal can be regarded as the sample signal and the water signal can be regarded as the volume signal. Alternatively, if the hemotocrit of the human body is known, the hemoglobin signal can be regarded as the volume signal and based on both hemoglobin and the hemotocrit, the amount of water can be determined. In addition, some FITC (Fluorosiein isothiocyauate) Fluoroscent marker with known concentration can be injected in.

To accurately measure these two signals, both glucose and volume signals are required to get from the same tissue. Particularly, if these signals are induced by an input signal, the input signal source(s) should be incident on the same tissue and then, the result data are collected from the targets through the collector. In the case of using an induced signal, there is a need to clamp the tissue that is to be excited. Such clamp, called “signal guide,” can be any structure that fixes the volume to be excited. The signal collector is used to fix the specific volume and time to collect signal for either enamation or induced signal.

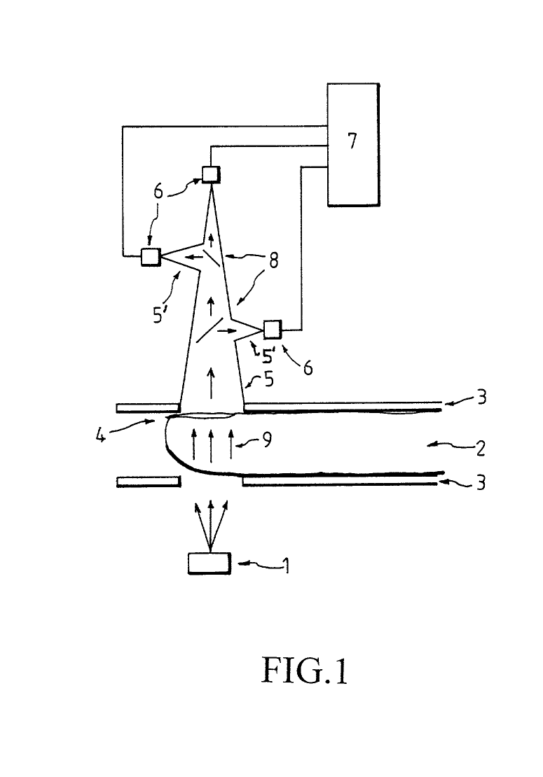

After the signals are collected, a spectroscopic method is needed to separate these two signals and collect the signals as many as possible. A conventional way is to use grating. According to the exemplary apparatus of the present invention shown in FIG. 1, two small cones 5′ and a large cone 5 housing two dichronic beam splitters 8 are used as the signal collector to ensure a better collection of signals from the tissue.

As shown in FIG. 1, the signals are collected from the finger 2. The light from the light source 1 is incident into the inner side of the finger 2 through a signal guide (not shown in the FIGURE). After being interactive with the finger 2, the light 9 comes out from the nail 4 side of the finger 2 and is collected by the cone 5. The finger 2 is clamped by an engulfed structure such as an envelope 3 to fix the position in the finger to be investigated. Both the signal guide and collector are attached to the envelope 3, so that the signal can came from the same piece of the sample.

In order to detect the concentrations of other ingredients in the blood, other specific signals, for example, signals of uric acid, cholesterol, triglycerol, oxyhemoglobin or any drugs or ingredients that are detected for their concentrations, are needed. Such signals can be detected one at a time by using the measurement apparatus shown in FIG. 1, by measuring a specific signal together with the signal of the solvent. Or, several ingredients (e.g., N−1 ingredients) can be detected at the same time. In the latter case, N−1 dichronic beam splitters are needed to separate N signals, and N cones (including 1 large cone and N−1 small cones), each of which has lens to collect and focus each of the N signals into corresponding designated detectors 6. The detectors 6, which are connected to the processing circuit 7, are set at the tips of the cones (5, 5′) so as to collect signals. A monochrometer that includes a band pass filter can be used to further refine the spectrum in each cone. The inner surfaces of the cones are made highly reflective to increase their ability to collect signals.

Instead, the signals could be enamations such as α, β or γ particles emitted from isotopes decay, or chemi-luminance-light emitted by chemical energy. The signals could also be secondary signals such as transmittance, scattering, fluorescence, Raman, etc., induced by another electromagnetic (EM) wave such as X-ray, visible, ultra-violet, infrared or microwave. To generate EM wave, all kinds of laser, diode laser, light emitted diode, lamps or EM sources can be used.

For any induced signals, there is always a time delay from excitation to emission of the induced signal. The incident signal could be guided at an earlier time to excite the target in a selected volume to be measured, and after time Δt, the induced signal is collected. This method is referred to as “time resolved technique.” The technique can be used in the exemplary apparatus for reducing noise. The technique will be more useful when the exited target is moving. Assume the target is at position x with a velocity V*. After Δt, the exited target will move to x+V*Δt and emits the induced signal at this position. The target can be exited in a volume at position x, as time t, then the induced signal from the target in the specific volume is measured at x+Δx=x+V*Δt, at the time t+Δt. Thus, the noise resulted from the stationary (not moving) parts can be cut.

The signal-noise ratio can be improved by further using switches. When the switch of the guide for the input signal is on, the switch for the collector is off; when the guide for the input signal is off, the switch for the collector is on. Such on-off circle can be repeated for a lot of times to improve the signal-noise ratio. The above-mentioned arrangement is very useful as the targets are moving in a conduit such as an artery or production line.

As the invention thus described, it will be obvious that the embodiments and description are not intended to limit the invention. The invention may vary in many ways. Such variations are not to be regarded as a departure from the spirit and scope of the invention, and all such modifications, as would be obvious to one skilled in the art, are intended for inclusion within the scope of the following claims.

We claim:

1. An apparatus for measuring the concentrations of (N−1) ingredients in a solution in-situ, wherein N is a natural number and N≧2,
said apparatus comprising:
a signal collector for collecting N signals from a target in a selected volume of the solution, one of said N signals being corresponding to said selected volume;
means for detecting said N signals; and
means for separating said N signals and transmitting said N separated signals to said detecting means.

2. The apparatus according to claim 1, wherein said N signals comprise at least one induced signal from said selected volume in response to an input signal.

3. The apparatus according to claim 2, wherein said input signal is in the form of electromagnetic wave.

4. The apparatus according to claim 1, wherein said signal collector comprises a plurality of cones for collecting said signals and/or for accommodating the transmission of said signals to said detecting means.

5. The apparatus according to claim 4, wherein said detecting means comprises a plurality of detectors respectively located at the tips of said plurality of cones.

6. The apparatus according to claim 1, wherein said separating means comprises a dichronic beam splitter.

7. The apparatus according to claim 1, wherein said separating means comprises N−1 beam splitters for separating said N signals.

8. The apparatus according to claim 7, wherein said signal collector comprises N cones for collecting said N signals.

9. The apparatus according to claim 5, wherein each of said plurality of cones comprises a lens for focusing the signal toward the corresponding detector.

10. The apparatus according to claim 4, wherein said plurality of cones comprise a highly reflective surface.

11. The apparatus according to claim 1, wherein said target comprises human tissue.

12. The apparatus according to claimed in claim 11, wherein said human tissue comprises a finger.

13. The apparatus according to claim 12, wherein said signal collector comprises an adapter located at the nail side of said finger for collecting said signals.

14. The apparatus according to claim 2, further comprising a signal guide for directing said input signal into said target.

15. The apparatus according to claim 14, wherein said signal guide directs said input signal into said target in said selected volume V at time t, and then said signal collector collects said signals from another selected volume V′, which V′ is the distribution of said target at time t=t+Δt.

16. The apparatus according to claim 15, wherein said target moves with a velocity V*, and said V′ is a linear transition from V to V+V*t.

17. The apparatus according to claim 16, wherein both said signal guide and signal collector respectively comprise a switch.

18. The apparatus according to claim 17, wherein the switch of said signal collector is open after a predetermined period of time when the switch of said signal guide is closed.

19. The apparatus according to claim 18, wherein said switches are changed between open and close for a plurality of times.

20. The apparatus according to claim 14, wherein said target comprises human tissue.

21. The apparatus according to claim 20, wherein said human tissue comprises a finger.

22. The apparatus according to claim 21, wherein said signal guide is at the inner surface of said finger.

23. The apparatus according to claim 22, further comprising an envelope for securing said finger.

24. The apparatus according to claim 13, further comprising an envelope for securing said finger.

25. The apparatus according to claim 1, wherein said signal corresponding to said selected volume is a signal corresponding to the solvent of said solution.

26. The apparatus according to claim 1, wherein said signal corresponding to said selected volume is a signal corresponding to a marker with known concentration.

27. The apparatus according to claim 25, wherein said solvent comprises water.

28. A spectrophotometer, comprising:
N cones for respectively collecting N signals;
N detectors for respectively detecting wave lengths of said N signals; and
N−1 dichronic beam splitters for respectively separating the spectrum of said N signals,
wherein, N is a natural number and N≧2.

29. The spectrophotometer according to claim 28, wherein said N detectors are respectively located at the tips of said N cones.

30. The spectrophotometer according to claim 28, further comprises a lens.

31. The spectrophotometer according to claim 28, wherein said N cones respectively comprise a highly reflective surface.

32. The spectrophotometer according to claim 28, further comprising a monochrometer for selecting the wave lengths.

33. A finger adapter for spectroscopic studying, comprising a collector for collecting a light emitted from a specific volume of a finger of a human body, and for directing said light toward a spectrophotometer.

34. The finger adapter according to claim 33, further comprising a guide at the inner surface of said finger, for guiding the light toward a definite volume of said finger.

35. The finger adapter according to claim 34, further comprising an envelope for securing said finger.

36. The finger adapter according to claim 35, wherein both said collector and guide respectively comprise a switch.

37. The finger adapter according to claim 36, wherein the switch of said collector is open after a predetermined period of time when the switch of said guide is closed.

38. The apparatus according to claim 37, wherein said switches are changed between open and close for a plurality of times.

39. The finger adapter according to claim 37, wherein said specific volume is at a distance of Δx=V*Δt from said definite volume, in which V* is the velocity of blood flow in said finger.
